(19) United States
(12) Patent Application Publication
(10) Pub. No.: US 2010/0119577 A1
(43) Pub. Date: May 13, 2010

(54) THERAPEUTIC COMPOSITE FOR CARTILAGE DISORDER USING EXTRACELLULAR MATRIX (ECM) SCAFFOLD

(75) Inventors: Byoung-Hyun Min, Seoul (KR); So Ra Park, Seoul (KR); Cheng Zhe Jin, Gyeonggi-do (KR); Byung Hyune Choi, Seoul (KR)

Correspondence Address: LUCAS & MERCANTI, LLP, 475 PARK AVENUE SOUTH, 15TH FLOOR, NEW YORK, NY, 10016, US

(73) Assignee: Byoung-Hyun MIN, Seoul (KR)

(21) Appl. No.: 12/598,523

(22) PCT Filed: May 6, 2007
(86) PCT No.: PCT/KR07/02215

§ 371 Date: Dec. 14, 2009

(51) Int. Cl. A61F 2/00; C12N 11/02; C12N 13/00; A61K 35/12

(52) U.S. Cl. 424/423; 435/177; 435/173.1; 424/93.7

(57) ABSTRACT

The present invention relates to a method for preparing a cell-derived ECM scaffold to which chondrocytes or stem cells are attached, a method for cartilage regeneration by tissue engineering, which comprises using the cell-derived ECM scaffold, and a therapeutic composition for treating cartilage disorder, which contains the ECM scaffold as an effective component. More specifically, the present invention relates to a method for cartilage regeneration by tissue engineering, which comprises transplanting ECM scaffold, having chondrocytes or stem cells attached thereto, into cartilage defects, and a therapeutic composition for treating cartilage disorder, which contains the ECM scaffold, having chondrocytes or stem cells attached thereto, as an effective component. According to the present invention, when the inventive ECM scaffold having chondrocytes or stem cells attached thereto is transplanted into a cartilage defect, mature articular cartilage having the same appearance and characteristics as those of natural cartilage tissue, can be regenerated without side effects such as inflammatory responses.

### TECHNICAL FIELD

The present invention relates to a method for preparing a cell-derived ECM scaffold to which chondrocytes or stem cells are attached, a method for cartilage regeneration by tissue engineering, which comprises the cell-derived ECM scaffold to which chondrocytes or stem cells are attached, and a therapeutic composition for treating cartilage disorder, which contains the ECM scaffold to which chondrocytes or stem cells are attached as an effective component.

### BACKGROUND ART

The method of autologous chondrocyte implantation (ACI) used for treating damaged cartilage is a clinically approved therapeutic method that can regenerate hyaline cartilage tissues in the defect (Brittberg, M. et al., New Eng. J. Med., 331:889, 1994). However, along with the development of studies on chondrocytes or mesenchymal stem cells (MSCs), more advanced methods are currently being developed including cell transplantation using various scaffolds and production of artificial cartilage tissues in vitro by tissue engineering (Lee, C. R., et al., Tissue Eng., 6:555, 2000; Li, W. J. et al., Biomaterials, 26:599, 2005).

Scaffolds, providing a 3D culture environment, affect not only proliferation and differentiation of inoculated cells but also the ultimate quality of tissue engineered cartilage. Currently, various substances originated from synthetic or natural materials are used for suitable scaffolds. Scaffolds are used in various forms such as sponge, gel, fiber, and micro beads, etc. (Honda, M. J., et al., J. Oral Maxillofac Surg., 62:1510, 2004; Griogolo, B. et al., Biomaterials, 22:2417, 2001; Chen, G. et al., J, Biomed. Mater. Res. A, 67:1170, 2003; Kang, S. W. et al., Tissue Eng., 11:438, 2005). Among them, the most commonly used type is a porous structure which can improve cell adhesion and maintain a high surface to volume ratio. Although some successful applications in vivo and in vitro were reported with some scaffolds, there was still a problem that they could not produce high-quality tissue engineered cartilage enough to be used in clinical practices. Therefore, to solve the problem, the scaffolds need to be improved in their structural and functional aspects.

Accordingly, the present inventors eventually expected that the production of hyaline cartilage tissue could be advanced by using ECM as a scaffold which is structurally complicated but composed of natural proteins and 3D structure of well-arrayed various macromolecules.

The ECM scaffold derived from chondrocytes fundamentally consists of type II collagen and glygosaminoglycan (GAG) that are principal components of cartilage tissue ECM, and also includes essential trace elements in chondrocyte metabolism. Since the ECM scaffold provides a natural environment for chondrocyte differentiation, it is needed to produce high quality scaffolds that can be applied to tissue engineering using ECM.

Articular cartilage damage is difficult to treat because it has limited intrinsic healing ability, and this easily causes degeneration of surrounding cartilage, thus resulting in extensive osteoarthritis (OA). It was reported that many surgical methods such as debridement, subchondral microfracture, and drilling help regeneration of damaged cartilage. However, the tissue regenerated using these methods generally has characteristics of fibrocartilage which lacks biomechanical characteristics of normal articular cartilage.

Due to incongruence of interface between transplant and host cartilage and restrictions in the donor source, there has been a limitation in applying the osteochondral transplantation techniques.

In 1994, Brittberg et al. reported an excellent result by using autologous chondrocyte transplantation (ATC) technique (Brittberg, M. et al., N. Engl. J. Med., 331:889, 1994). However, there are still some remaining problems with the method, which include loss of chondrocytes function during in vitro culture, the possibility of transplanted chondrocytes leaking from the transplant site, heterogeneous distribution of chondrocytes, and periosteal hypertrophy. Therefore, many researchers are seeking biocompatible substances for tissue engineering to effectively deliver chondrocytes to lesions. Until now, many biocompatible substances, both of the natural (e.g. fibrin and collagen) and the synthetic (e.g. polyglycolic acid, polylactic acid, polylactic-glycolic acid), have been used for cartilage tissue engineering. Regrettably, however, no biocompatible substance has been reported that showed satisfying results in the production of mature cartilage in vivo.

Accordingly, the present inventors have made extensive efforts to develop a novel biocompatible substance, and as a result, confirmed that when a chondrocyte-derived ECM scaffold having chondrocytes or mesenchymal stem cells attached thereto, is transplanted into a cartilage defect, mature articular cartilage having the same morphology and characteristics as the native cartilage is regenerated, thereby completing the present invention.

### SUMMARY OF THE INVENTION

Therefore, the main object of the invention is to provide a method for producing a cell-derived ECM scaffold having chondrocytes or stem cells attached thereto.

Another object of the present invention is to provide a therapeutic composition for treating cartilage disorder, which contains the ECM scaffold having chondrocytes or stem cells attached thereto as an effective component.

To achieve the above objects, the present invention provides a method for preparing a cell-derived ECM scaffold for treating cartilage disorder, to which chondrocytes or stem cells are attached, the method comprises inoculating animal chondrocytes or stem cells into the cell-derived ECM scaffold, and then culturing it.

The present invention also provides a method for cartilage regeneration by tissue engineering, the method comprises transplanting the ECM scaffold having chondrocytes or stem cells are attached thereto, which is prepared by the above method, into cartilage defects in animals.

The present invention also provides a therapeutic composition for treating cartilage disorder, which contains the ECM scaffold having chondrocytes or stem cells attached thereto, as an effective component.

Another features and embodiments of the present invention will be more clarified from the following “detailed description” and the appended “claims”.

### BRIEF DESCRIPTION OF DRAWINGS

FIG. 1 shows the results of safranin-O staining of transplant samples cultured in vitro at various incubation periods, according to the present invention.

FIG. 2 shows the results of measuring DNA (A), GAG (B), and collagen (C) content of transplant samples cultured in vitro at various incubation periods, according to the present invention.

FIG. 3 shows the results of measuring compressive strength of transplant samples cultured in vitro according to the present invention.

FIG. 4 is photographs taken right after transplanting the inventive transplant into cartilage defect in vivo.

FIG. 5 is photographs of condyles collected at 1 month and 3 months after transplanting the inventive transplants into the cartilage defect in vivo, which was observed by naked eye.

FIG. 6 is photographs of repaired cartilage stained with safranin-O at 1 month and 3 months after transplanting (A, C, E, G, I, K, M, and O: x 40; B, D, F, H, J, L, N, and P: x 100).

FIG. 7 is a graph that shows ICRS scores according to the results of safranin-O staining of repaired cartilage at 1 month and 3 months after transplanting.

FIG. 8 is the results of immunohistochemical staining showing the expression collagen type II in repaired cartilage at 1 month and 3 months after transplanting.

FIG. 9 shows the results of safranin-O staining according to incubation period of the inventive scaffold, to which mesenchymal stem cells are attached in vitro.

FIG. 10 is photographs of transplants obtained after in vivo transplantation of ECM scaffold having mesenchymal stem cells attached thereto and PGA scaffold having mesenchymal stem cells attached thereto as a control, which was observed by naked eye.

FIG. 11 is graphs that show changes in the size of transplants obtained after in vivo transplantation of ECM scaffold having mesenchymal stem cells attached thereto and PGA scaffold having mesenchymal stem cells attached thereto.

### DETAILED DESCRIPTION OF THE INVENTION, AND PREFERRED EMBODIMENTS

In one aspect, the present invention relates to a method for preparing a cell-derived ECM scaffold, to which chondrocytes or stem cells are attached, the method comprises inoculating animal chondrocytes or stem cells into the cell-derived ECM scaffold, and then culturing it.

In the present invention, growth factors or cytokine can be additionally added to the step of culturing the chondrocytes or stem cells, and as growth factors added, IGF (insulin-like growth factor), FGF (fibroblast growth factor), TGF (tissue growth factor), BMP (bone morphogenetic proteins), NGF (Nerve growth factor), PDGF (platelet-derived growth factor), or TNF-α can be used, but not limited thereto.

In the present invention, the cell-derived ECM scaffold is prepared by the following steps: (a) culturing chondrocytes isolated from animal cartilage, and then obtaining a chondrocytes/extracellular matrix (ECM) membrane from the cultured chondrocytes; (b) obtaining a scaffold-free, pellet-type construct by culturing the obtained chondrocytes/ECM membrane; and (c) obtaining an ECM scaffold by freeze-drying the obtained pellet-type construct, but not limited thereto.

In the present invention, the ECM scaffold can be prepared by additionally performing the step (d) of decellularizing the ECM scaffold, said step of decellularizing is carried out by treating the ECM scaffold with any one selected from the group consisting of protease, detergent, DNase and ultrasound.

The prepared ECM scaffold may be processed to have a desired form by molding it after powderization.

In the present invention, the step (c) is preferably performed by repeating cycles of freezing at −15° C. to −25° C. and thawing the obtained pellet-type construct 3-5 times, and then freeze-drying it.

As animals used in the present invention, any animal can be used without limitations as long as it is a vertebrate having cartilages, and preferably pig, rabbit, mouse, rat, dog, calf, goat, and cat can be used.

Meanwhile, when the stem cells are treated with ultrasound or mechanical stimulus such as a physical pressure thereto during culture, differentiation into chondrocytes can be promoted.

After transplanting chondrocytes or stem cells grown in the ECM scaffold of the present invention, it was found that higher quality cartilage tissue was regenerated, as the incubation period of the constructs in vitro became longer. Therefore, the culture in vitro is preferably performed for 1-10 weeks, and more preferably performed for 3-8 weeks.

In the present invention, the chondrocytes may be autologous cells, or xenogeneic or homologous cells, the stem cells may include cells selected from the group consisting of mesenchymal stem cells, hematopoietic stem cells, fetal stem cells, adipose stem cells, umbilical cord blood stem cells, and embryonic stem cells.

In the present invention, the mesenchymal stem cells are preferably derived from the bone marrow, embryo, fetus, cord blood, synovium, myoblasts, amniotic fluid, adipocytes, or adult tissue.

In another aspect, the present invention relates to a method for cartilage regeneration by tissue engineering, the method comprises transplanting the ECM scaffold, to which chondrocytes or stem cells are attached, into cartilage defects in animals.

When the transplant site is treated with ultrasound or mechanical stimulus such as a physical pressure thereto, regeneration of chondrocytes can be promoted.

In still another aspect, the present invention relates to a therapeutic composition for treating cartilage disorder, which contains the ECM scaffold prepared by the above method, to which chondrocytes or stem cells are attached, as an effective component.

In the present invention, the cartilage disorder is preferably selected from the group consisting of degenerative arthritis, rheumatoid arthritis, fractures and disc disease.

After inoculating chondrocytes or stem cells grown in monolayer culture of the present invention into an ECM scaffold, the ECM scaffold having chondrocytes or mesenchymal stem cells attached thereto, which is cultured for 4 weeks in vitro (hereinafter referred to as transplant), was transplanted into a cartilage defect, thus confirming that 3 months after the transplantation, mature cartilage tissue, which is similar to the surrounding natural cartilage, was generated.

In addition, it was also confirmed that the inventive ECM scaffold, having chondrocytes or mesenchymal stem cells attached thereto, did not cause any inflammatory response after transplantation in vivo.

### Examples

Hereinafter, the present invention will be described in further detail with reference to examples. It is to be understood, however, that these examples are for illustrative purposes only and are not to be construed to limit the scope of the present invention.

Especially, in the following examples, cartilage was regenerated by transplanting an ECM scaffold having chondrocytes attached thereto into cartilage defects of rabbits. However, it is obvious to those skilled in the art that similar effects can be obtained by transplanting an ECM scaffold, having stem cells capable of differentiating into chondrocytes attached thereto, into cartilage defects.

### Example 1

### Construction of Cell-Derived ECM Scaffold

A cell-derived ECM scaffold was constructed using chondrocytes isolated from knee joints of 2 to 4-week-old pigs.

After culturing pig chondrocytes for 3˜4 days, cell layers having ECM components, were carefully separated, and transformed into a pellet-type construct through centrifugation. The pellet-type structure was adjusted for 3 weeks for the growth of new cartilage tissue, after repetition of freeze-thawing 3 times every 12 hours, ECM was obtained by freeze-drying at −56° C. for 48 hours at a pressure of 5mTorr.

The constructed ECM was decellularized through a step of removing nuclear and cytoplasmic components by treating it with a proteolytic enzyme, detergent or ultrasound. 0.05% trypsin was used as a proteolytic enzyme and ionic and non-ionic detergents such as SDS, triton X, deoxycholate were used as a detergent. Nuclear components, such as DNA, etc. were removed by treating it with DNase.

The final decellularized ECM was ground into fine powder, and dispersed and dissolved in an acid solution (pH 3), then formed into a desirable form using a mold, followed by modifying it using chemical cross-linking such as EDC/NHS method, thus constructing an ECM scaffold, and finally the structure was trimmed off by a thickness of less than 1 mm.

### Example 2

### Construction of Tissue-Engineered Cartilage Using Rabbit Chondrocytes and ECM Scaffold

The cell-derived ECM scaffold constructed in Example 1 was socked in 70% ethanol for 1 hour to wash with PBS several times, and left to stand overnight in DMEM medium without serum before inoculating cells. Chondrocytes were isolated from a 2 week-old New Zealand white rabbit and cells at passage 1 were inoculated into the ECM scaffold at a concentration of 3×10⁶ cells/ml for 1.5 hours. The ECM scaffold inoculated with chondrocytes was cultured in a 6-well plate for 2 days, 2 weeks and 4 weeks, before transplantation.

### Example 3

### Maturity Measurement of Transplant In Vitro

### 3-1: Histological and Immunohistochemical Analysis

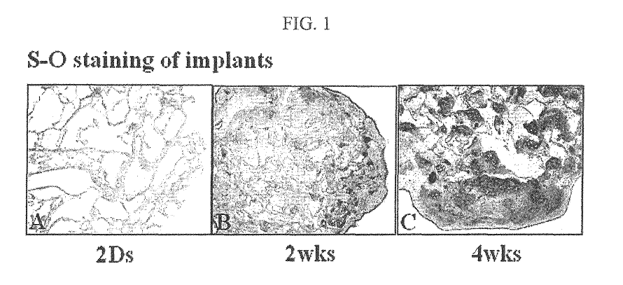

The cultured ECM scaffold containing chondrocytes, constructed in Example 2, was fixed in 4% formalin fixative solution for 24 hours. Then, the sample was embedded in paraffin, sectioned into a thickness of 4 μm, and stained with safranin-O. As a result, it was observed that accumulation of sulfated proteoglycan was increased gradually with time, which filled the pores of the scaffold (FIG. 1).

For chemical analysis, the transplant (the cultured ECM scaffold containing chondrocytes) was decomposed with a papain solution (125 μg/ml of papain, 5 mM of L-cystein, 100 mM of Na2HPO4, 5 mM of EDTA, pH 6.4) at 60° C. for 24 hours and centrifuged at 12,000 g for 10 minutes. For the measurement of total GAG (glycosaminoglycan) content, culture supernatant was analyzed using DMB assay (1,9-dimethylmethylene blue). Each sample was mixed with DMB solution to measure the absorbance at 225 nm. Total GAG of each sample was quantified by a standard curve using 0˜5 μg/ml shark condroitin sulfate (Sigma, USA).

The collagen content was measured using hydroxyproline analysis. Bovine collagen (0˜10 μg/ml, Sigma, USA) was used as a standard solution. The DNA content was measured using Quant-iT™ dsDNA BR assay kit (Invitrogen, USA). ECM scaffolds, which are not inoculated with chondrocytes, were used as a control group in all analysis.

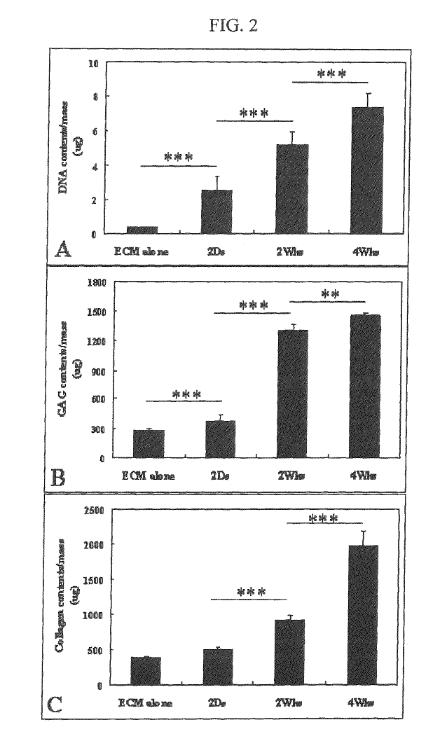

As a result, the DNA content of the transplant was remarkably increased from day 2, and it was more than three times at week 4. The GAG content was especially increased from week 2 (FIG. 2), it was 276.5±20.6 μg in a control group treated only with an ECM scaffold, 378.5±65.6 μg in a group cultured for 2 days, 1302.8±65.4 μg in a group cultured for 2 weeks, and 1450±30 μg in a group cultured for 4 weeks.

The collagen content was also increased gradually with time, and it was shown that the collagen content at week 4 increased more than 4 folds compared with the collagen content at week 2 (FIG. 2C). The amount of GAG newly synthesized by rabbit chondrocytes, was 1173.5 μg at week 4, and collagen content was 1587.9 μg.

In the present examples, one-way analysis (ANOVA) was used for multiple comparisons of experimental data, and student t test (two tails) was used for a comparison of matched pairs. Statistical significance was represented as *p<0.05, **p<0.01 and ***p<0.001.

### 3-2: Measurement of Mechanical Compressive Strength of Transplant

Mechanical compressive strength of the cultured ECM scaffold containing chondrocytes (transplant), which is constructed in Example 2, was measured using Universal Testing Machine (Model H5K-T, H.T.E., England). Samples (n=5) cut into a regular disc form were placed on a metal plate, and the strength was measured at a crosshead speed of 1 mm/min and 0.001 preload was applied thereto. Compressive strength of each sample was calculated at a point where it was compressed to 10% strain, natural cartilage isolated from a 2 week-old rabbit, was used as a control group.

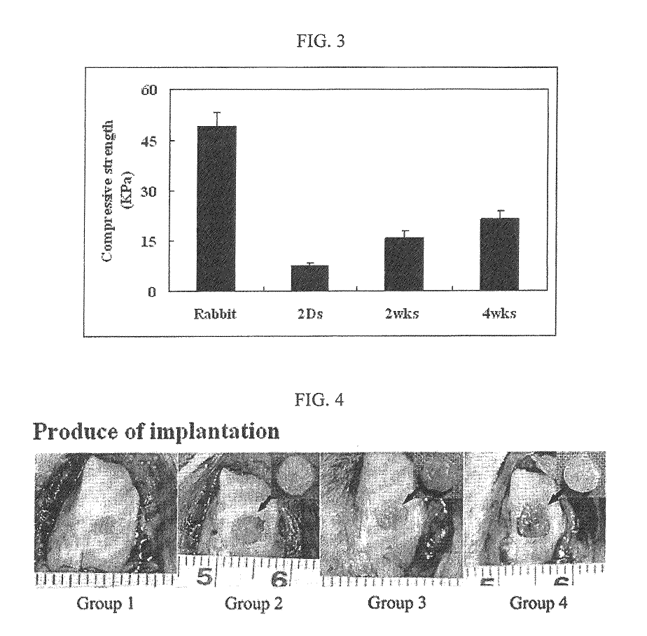

As a result, compressive strength of transplant cultured for 2 days was 0.7±0.08 KPa, compressive strength of transplant cultured for 2 weeks was 16.4±2.2 KPa, compressive strength of transplant cultured for 4 weeks was 21.5±2.2 KPa (FIG. 3). Compressive strength of transplant cultured for 4 weeks was 30 folds higher than that of transplant cultured for 2 weeks, suggesting that compressive strength of a transplant increases with incubation time in vitro. Compressive strength (21.5±2.2 KPa) of transplant cultured for 4 weeks was 44% of the compressive strength of natural rabbit cartilage (49±2.2 KPa).

### Example 4

### In Vivo Transplantation and Analysis Thereof

### 4-1: In Vivo Transplantation of Transplant

11 New Zealand white rabbits weighing average 3.0 to 3.5 kg were used. After anesthesia using ketamine and lumpun at a ratio of 3 to 1.5, surgical treatment procedure was performed, and both knee joints underwent identical operations. To expose the femoral condyle of the knee joint, a vertical midline incision was made in the joint capsule adjacent to the patella which will be dislocated, and osteochondral defect was made on the patellar groove by using a 5 mm drill.

Total 36 of knee joints were divided into untreated control group (group 1), a group transplanted with a scaffold cultured for 2 days in vitro (group 2), a group transplanted with a scaffold cultured for 2 weeks (group 3) and a group transplanted with a scaffold cultured for 4 weeks (group 4). Transplants were pressure-fixed without cover slips or suture material after they were inserted into defects (FIG. 4).

### 4-2: Collecting Transplants and Observation of Cartilage Defects by Naked Eye

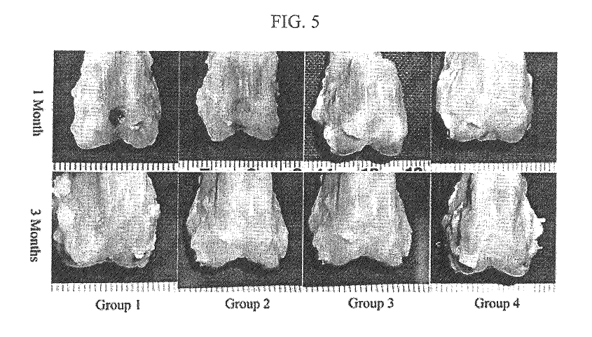

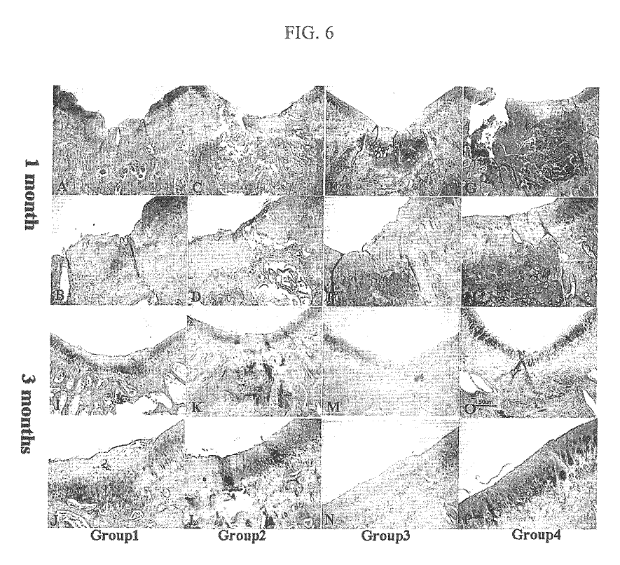

1 month and 3 months after operation, rabbits were euthanized by injecting an excessive dose of pentobarbital, and the femoral condyle was collected to observe cartilage defects by naked eye. As a result, as shown in FIG. 5, transplants were stably inserted into the defects. At 1 month after operation, the repaired defects had smooth and glossy appearance, and exhibited continuity with the surrounding host cartilage in group 3 and group 4.

On the contrary, it was observed that the defect was not repaired in group 1, and the defect was partially filled with fibrous tissue in group 2. 3 months after operation, repaired tissue having white and glossy appearance was observed in defects of all groups. However, repaired tissue having smooth and hard surface was observed in group 2, group 3 and group 4 by forceps, while rough surface and many cracks were observed in group 1.

### 4-3: Histological Characteristics

Sample was embedded in paraffin, micro-cross sectioned, and stained with safranin-O to observe histologically. As a result, 1 month after operation, the defects of group 1 and group 2 were partially filled with fibrous tissue, and were not connected to the surrounding cartilage and bone tissue (FIG. 6A˜D). On the contrary, hyaline cartilage-like tissue was partially observed in group 3, and the complete regeneration of hyaline cartilage was observed in group 4 (FIG. 6 E˜H). Repaired tissue was successfully integrated with the host cartilage, but subchondral bone was not regenerated in all groups. Fibrous/hyaline cartilage was regenerated and partially integrated with the surrounding host cartilage in group 1, group 2 and group 3 of samples at 3 months after operation (FIG. 6I˜N). Among the groups, the surface of repaired tissue was rough in group 1, and subchondral bone was partially regenerated in group 1, group 2 and group 3 while hyaline cartilage tissue having mature matrix and cylindrical cartilaginous tissue was observed, and subchondral bone was completely regenerated in group 4 (FIG. 6O˜P).

To measure the degree of articular cartilage regeneration in defects by ICRS score, it was graded using modified histological grading criteria. The grading system is based on seven criteria with scores ranging from 0 to 18 (Table 1).

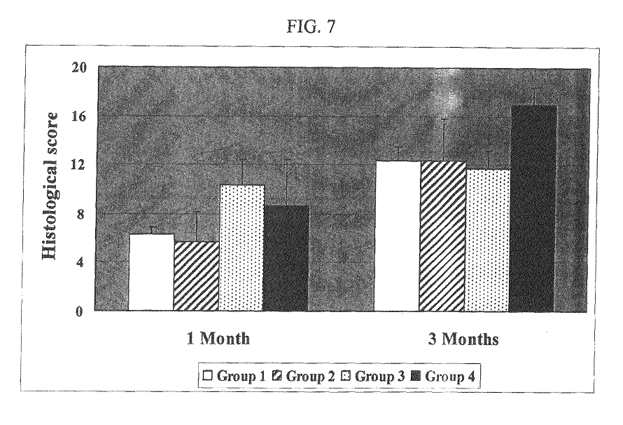

In all groups, ICRS score significantly increased with time (FIG. 7), ICRS scores in groups 3 and 4 were higher than those in groups 1 and 2 at 1 month after operation, and ICRS score of group 4 was the highest at 3 months after operation.

TABLE I
Histological scoring system

| Category | Points |
|---|---|
| Cell morphology | |
| Hyaline cartilage | 4 |
| Mostly hyaline cartilage | 3 |
| Mostly fibrocartilage | 2 |
| Mostly non-cartilage | 1 |
| Non-cartilage only | 0 |
| Matrix-staining (metachromasia) | |
| Normal | 3 |
| Slightly reduced | 2 |
| Markedly reduced | 1 |
| No metachromatic staining | 0 |
| Structural integrity | |
| Normal | 2 |
| Slight disruption | 1 |
| Severe disintegration | 0 |
| Surface regularity | |
| Smooth | 3 |
| Moderate | 2 |
| Irregular | 1 |
| Severely irregular | 0 |
| Thickness of cartilage (%) | |
| 121-150 | 1 |
| 81-120 | 2 |
| 51-80 | 1 |
| 0-50 | 0 |
| Regenerated subchondral bone | |
| Good | 2 |
| Moderate | 1 |
| Poor | 0 |
| Integration with adjacent cartilage | |
| Both edges integrated | 2 |
| One edge integrated | 1 |
| Neither edge integrated | 0 |
| Total maximum | 18 |

### 4-4: Analysis of Collagen Type II Expression

To examine expression of collagen type II in each transplant, immunohistochemical analysis was performed. Specimens were allowed to react with mouse monoclonal anti-rabbit collagen type II antibody (1:200, Chemicon, USA) for 1 hour and additionally reacted with biotnylated secondary antibody (dilution 1:200) for 1 hour. Finally, the specimen was allowed to react with peroxidase conjugated strepavidin solution (DAKO LSAB system, USA) for 30 minutes. The immunostained specimen was counter-stained with Mayer's hematoxylin (Sigma, USA) and observed with a microscope (Nikon E600, Japan).

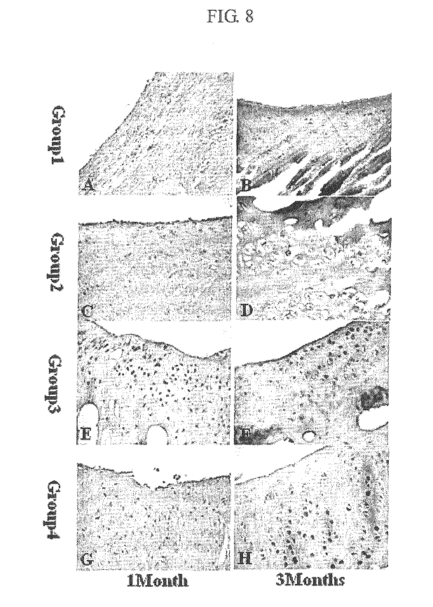

As a result, the expression of collagen type II was gradually increased in the pericellular area of repaired tissue with time in group 2, group 3 and group 4, but it was not particularly observed in group 1 both at 1 month and 3 months after operation (FIG. 8). At 3 months after operation, the expression of the collagen type II with a dark brown colour was observed most clearly at zonal structure of group 4 (FIG. 8H).

### Example 5

### Construction of Tissue-Engineered Cartilage Using Rabbit Mesenchymal Stem Cells and ECM Scaffold

Mesenchymal stem cells were isolated from 2-week-old female New Zealand white rabbits (Central center of experimental animals, Korea). Bone marrow aspirate isolated from tibia and femur was suspended in 5% acetic acid, and centrifuged for 5 minutes at 1,500 rpm to remove erythrocytes, thus obtaining mesenchymal stem cells.

The mesenchymal stem cells were suspended in α-MEM (Minimum essential medium, alpha modification of Eagle's medium; Sigma, USA) containing antibiotics and 10% NCS (new-born calf serum), and dispensed in a tissue culture plate at a concentration of 1.5×10⁷ to culture in 5% CO₂ incubator at 37° C. for 2 weeks.

14 days after incubation, primary cultured cells were treated with 0.05% trypsin-EDTA (Gibco-BRL Life Technologies, USA), and centrifuged at 1,500 rpm for 5 minutes to form pellets. Cell pellets were plate-cultured at a density of 1.5×10⁶ cells/plate, and culture medium was replaced three times a week. Secondary sub-cultured cells were injected into the ECM scaffold prepared in Example 1 and PGA (polyglycolic acid) scaffold (control group), and cultured for 1 week, 2 weeks and 4 weeks to induce differentiation, thus performing analysis of their appearance and histological analysis by the same method as 3-1 of Example 3.

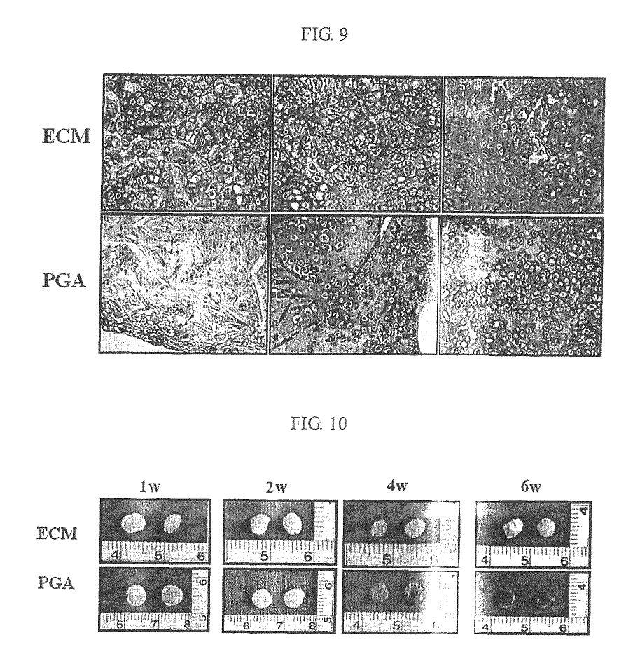

As a result of examining the amount of GAG expressed using safranin-O staining, GAG was highly expressed in the ECM scaffold starting from 1 week after incubation, and high levels of GAG expression were observed in both control group (PGA scaffold) and experimental group (ECM scaffold) at 4 weeks after incubation (FIG. 9).

As a result of examining GAG expression and observing by naked eye, it was confirmed that the differentiation of rabbit mesenchymal stem cells into chondrocytes was effectively induced in the ECM scaffold.

### Example 6

### In Vivo Transplantation of Scaffold Having Mesenchymal Stem Cells Attached Thereto and Analysis Thereof

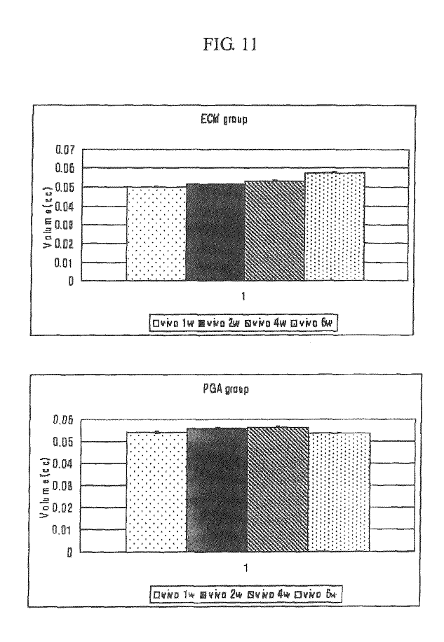

ECM scaffolds having rabbit mesenchymal stem cells attached thereto, in which differentiation was induced for 1 week by the method of example 5, and PGA scaffolds having rabbit mesenchymal stem cells attached thereto were transplanted into nude mice, and the nude mice were observed for 1 week, 2 weeks, 4 weeks and 6 weeks. As a result of observing morphology, the size of the scaffolds was decreased in both groups with the passage of time, but no significant changes were observed. However, in the case of the group with PGA scaffolds, red color was exhibited due to high blood vessel penetration thereof, while the group with ECM scaffolds showed relatively low blood vessel penetration (FIG. 10).

As a result of measuring changes in the size of the scaffolds in the two groups with the passage of time, there was no significant changes overall. However, it was shown that the size of ECM scaffold tends to increase continuously, while that of PGA scaffold tends to decrease starting at week 6 after incubation (FIG. 11).

### INDUSTRIAL APPLICABILITY

As described above in detail, the present invention has an effect of providing therapeutic composite for treating cartilage disorder, which contains an ECM scaffold having chondrocytes or stem cells attached thereto as an effective component. When the inventive ECM scaffold having chondrocytes or stem cells attached thereto is transplanted into a cartilage defect, mature articular cartilage having the same appearance and characteristics as those of natural cartilage tissue, can be regenerated without side effects such as inflammatory responses.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for preparing a cell-derived ECM scaffold, to which chondrocytes or stem cells are attached, the method comprises inoculating animal chondrocytes or stem cells into the cell-derived ECM scaffold, and then culturing it.

2. The method for preparing a cell-derived ECM scaffold according to claim 1, wherein the cell-derived ECM scaffold is prepared by the following steps:
(a) culturing chondrocytes isolated from animal cartilage, and then obtaining a chondrocytes/extracellular matrix (ECM) membrane from the cultured chondrocytes;
(b) obtaining a scaffold-free, pellet-type construct by culturing the obtained chondrocytes/ECM membrane; and
(c) obtaining an ECM scaffold by freeze-drying the obtained pellet-type construct.

3. The method for preparing a cell-derived ECM scaffold according to claim 2, which additionally comprises the step (d) of decellularizing the ECM scaffold.

4. The method for preparing a cell-derived ECM scaffold according to claim 3, wherein decellularizing is carried out by treating the ECM scaffold with any one selected from the group consisting of protease, detergent, DNase and ultrasound.

5. The method for preparing a cell-derived ECM scaffold according to claim 2, which additionally comprises powderizing the obtained ECM scaffold, and then forming it.

6. The method for preparing a cell-derived ECM scaffold according to claim 3, which additionally comprises powderizing the obtained ECM scaffold, and then forming it.

7. The method for preparing a cell-derived ECM scaffold according to claim 2, wherein the step (c) comprises repeating cycles of freezing at −15 to −25° C. and thawing the obtained pellet-type construct 3-5 times, and then freeze-drying it.

8. The method for preparing a cell-derived ECM scaffold according to claim 1, wherein said chondrocytes are autologous cells, or xenogeneic or homologous cells.

9. The method for preparing a cell-derived ECM scaffold according to claim 1, wherein said stem cells are selected from the group consisting of mesenchymal stem cells, hematopoietic stem cells, fetal stem cells, adipose stem cells, umbilical cord blood stem cells and embryonic stem cells.

10. The method for preparing a cell-derived ECM scaffold according to claim 1, wherein said culturing is performed for 1 day to 10 weeks.

11. The method for preparing a cell-derived ECM scaffold according to claim 1, wherein growth factors or cytokine are additionally added in the culturing step.

12. The method for preparing a cell-derived ECM scaffold according to claim 11, wherein said growth factor is selected from the group consisting of IGF (insulin-like growth factor), FGF (fibroblast growth factor), TGF (tissue growth factor), BMP (bone morphogenetic proteins), NGF (Nerve growth factor), PDGF (platelet-derived growth factor) and TNF-α.

13. The method for preparing a cell-derived ECM scaffold according to claim 1, wherein culture broth is treated with ultrasound or a mechanical stimulus is applied thereto during culture.

14. A method for cartilage regeneration by tissue engineering, the method comprises transplanting the ECM scaffold having chondrocytes or stem cells attached thereto, which is prepared by the method of claim 1, into cartilage defects in animals.

15. A therapeutic composition for treating cartilage disorder, which contains the ECM scaffold having chondrocytes or stem cells attached thereto, which is prepared by the method of claim 1, as an effective component.

16. The therapeutic composition for treating cartilage disorder according to claim 15, wherein the cartilage disorder is selected from the group consisting of degenerative arthritis, rheumatoid arthritis, fracture and disc disease.
